(19) United States
(12) Patent Application Publication
(10) Pub. No.: US 2020/0016277 A1
(43) Pub. Date: Jan. 16, 2020

(54) NANOPARTICLES FOR ACTIVE AGENT DELIVERY TO BRAIN CANCERS

(71) Applicant: MUSC FOUNDATION FOR RESEARCH DEVELOPMENT, Charleston, SC (US)

(72) Inventors: Ann-Marie BROOME, Mount Pleasant, SC (US); Suraj DIXIT, James Island, SC (US); Amy-Lee BREDLAU, Mount Pleasant, SC (US)

(21) Appl. No.: 16/485,518

(22) PCT Filed: Feb. 23, 2018
(86) PCT No.: PCT/US2018/019373

§ 371 Date: Aug. 13, 2019

Related U.S. Application Data
(60) Provisional application No. 62/462,421, filed on Feb. 23, 2017.

(51) Int. Cl. A61K 47/69; A61K 47/62; A61P 35/00; A61K 31/495

(52) U.S. Cl. CPC A61K 47/6909; A61K 31/495; A61P 35/00; A61K 47/62

(57) ABSTRACT

The present invention is directed to targeted micelle active agent carriers. The carriers suitably include micelle forming components, along with pH sensitive molecules, and targeting moieties. They are useful in the treatment of various brain cancers.

### BACKGROUND OF THE INVENTION

### Field of the Invention

The present invention is directed to targeted micelle active agent carriers. The carriers suitably include micelle forming components, along with pH sensitive molecules, and targeting moieties. They are useful in the treatment of various brain cancers.

### Background of the Invention

Brain cancer is a life-threatening disease in which only a minority of patients is likely to survive (overall 5-year relative survival rate for 2005-2011 was 33.3%, NIH NCI). Late diagnosis and limitations of conventional therapies, as a result of inefficient delivery, non-specificity to brain tumors and chemo-resistance, are among the major reasons for this poor prognosis. Nanoparticle (NP) drug vehicles provide a promising platform technology that can allow for targeted delivery of combined diagnostic and therapeutic agents for cancer treatment. What is needed is a therapeutic NP delivery vehicle which can provide specific tumor targeting and an increased therapeutic index allowing for the treatment and post-therapy monitoring of brain cancers, while minimizing side effects. The present invention meets these needs.

### BRIEF SUMMARY OF THE INVENTION

In embodiments, provided herein are targeted micelle active agent carriers. Such carriers suitably include a micellar structure comprising a poly(ethylene glycol)-lipid (PEG-lipid) and a pH sensitive molecule, a targeting moiety associated with the PEG-lipid; and an active agent encapsulated within the micellar structure.

Exemplary PEG-lipids include PEG-phosphatidylethanolamine-amine (PEG-PE-amine), while suitable pH sensitive molecules include N-palmitoyl homocysteine (PHC) and D-α-tocopheryl polyethylene glycol succinate (TPGS). The micellar structure can also include a phosphatidylcholine lipid and cholesterol.

In embodiments, the targeting moiety targets a receptor tyrosine kinase (RTK) receptor, and can be a platelet-derived growth factor (PDGF) peptide or an epidermal growth factor (EGF) peptide.

Suitably, the active agent is a chemotherapeutic agent, for example temozolomide.

In embodiments, targeting moiety and the micellar structure are present at a molar ratio of about 0.005 to about 0.01 (targeting moiety:micellar structure).

Also provided are methods of treating a brain cancer in a patient, which include administering the targeted micelle active agent carrier described herein to the patient, wherein the targeted micelle active agent carrier crosses the blood-brain barrier to target the brain cancer and deliver the active agent, thereby treating the brain cancer. In embodiments, the brain cancer is a glioblastoma.

In further embodiments, provided herein are targeted micelle active agent carriers, which include a micellar structure comprising poly(ethylene glycol)-phosphatidylethanolamine-amine (PEG-PE-amine) and D-α-tocopheryl polyethylene glycol succinate, a targeting moiety associated with the PEG-PE-amine, and an active agent encapsulated within the micellar structure. Suitably, the targeting moiety targets a receptor tyrosine kinase (RTK) receptor, and is a platelet-derived growth factor (PDGF) peptide or an epidermal growth factor (EGF) peptide. In embodiments, the active agent is a chemotherapeutic, such as temozolomide. The micellar structure can further include a phosphatidylcholine lipid and cholesterol, and suitably the targeting moiety and the micellar structure are present at a molar ratio of about 0.005 to about 0.01 (targeting moiety: micellar structure).

Also provided herein is the use of the targeted micelle active agent carrier as described herein for the treatment of a glioblastoma in a patient, wherein the targeted micelle active agent carrier crosses the blood-brain barrier to target the glioblastoma and deliver the active agent. Suitably, the targeting moiety is a platelet-derived growth factor (PDGF) peptide and wherein the targeting moiety and the micellar structure are present at a molar ratio of about 0.008 to about 0.01 (targeting moiety:micellar structure).

Further embodiments, features, and advantages of the embodiments, as well as the structure and operation of the various embodiments, are described in detail below with reference to accompanying drawings.

### BRIEF DESCRIPTION OF THE DRAWINGS/FIGURES

FIG. 1 shows a schematic drawing of a targeted micelle active agent carrier, in accordance with an embodiment hereof.

FIG. 2 shows micelle concentrations using ultraviolet-visible spectroscopy of free temozolomide (TMZ), micelle temozolomide (MTMZ) and targeted micelle temozolomide (PMTMZ).

FIG. 3 shows size calculation using dynamic light scattering of MTMZ (untargeted) and PMTMZ (targeted).

FIG. 4 shows intensity of TMZ (325 nm)-filled nanoparticles between pH 4 and 10, illustrating the loss of micellar contents outside of the physiologic range due to rupture.

FIG. 5 shows stability of MTMZ and PMTMZ over time in phosphate-buffered saline. Both micelles were able to maintain their composition over a 24-h period.

FIG. 6 shows stability of MTMZ and PMTMZ over time in serum. Both micelles were able to maintain their composition over a 24-h period.

FIG. 7 shows transmission electron microscopy of PMTMZ, illustrating spherical micelles with a diameter of approximately 12-13 nm.

FIGS. 8A-8D show uptake of both MTMZ and PMTMZ by glioma cells.

FIGS. 9A-9B show evaluation of kinetic-based uptake of MTMZ and PMTMZ, with mean fluorescence imaging of internalized micelles.

FIG. 10 shows inhibition of receptor-mediated uptake using brefeldin.

FIG. 11A shows cell toxicity and death of U87 cells treated with PDGFR-micelles containing TMZ (10 μM) versus micelle-encapsulated TMZ (10 μM) and free TMZ (10 or 100 μM).

FIG. 11B shows cell toxicity and death of U87 cells treated with 1 μM TMZ, as free drug, and in targeted and untargeted micelles.

FIGS. 12A-12B show accumulation of PDGF-micelles containing temozolomide in orthotopic gliomas in mice with orthotopically implanted with U87-luciferase cells in the left hemisphere of the brain.

FIG. 12C shows relative fluorescence quantified over time from a region of interest indicating the brain tumor. Error bars represent standard deviation.

FIG. 12D shows micelle fluorescence observed in excised mouse brains from respectively treated animals using an in vivo fluorescence imaging system.

FIGS. 12E-12I show biodistribution of the PMTMZ imaged over the 24-h period in mice placed to show ventral organs (n=4 per group). Fluorescence intensity decreases over time postinjection.

FIG. 13 shows dynamic light scattering results illustrating the size of untargeted (PEM) and targeted (PMTMZ) TMZ-containing micelles.

FIG. 14 shows the absorbance spectrum of untargeted and targeted, TMZ-containing, micelles.

FIG. 15 shows stability of targeted TMZ-containing micelles.

### DETAILED DESCRIPTION OF THE INVENTION

It should be appreciated that the particular implementations shown and described herein are examples and are not intended to otherwise limit the scope of the application in any way.

The published patents, patent applications, websites, company names, and scientific literature referred to herein are hereby incorporated by reference in their entireties to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

As used in this specification, the singular forms “a,” “an” and “the” specifically also encompass the plural forms of the terms to which they refer, unless the content clearly dictates otherwise. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

Technical and scientific terms used herein have the meaning commonly understood by one of skill in the art to which the present application pertains, unless otherwise defined. Reference is made herein to various methodologies and materials known to those of ordinary skill in the art.

### Challenges of Treating Brain Cancer

Glioblastoma multiforme (GBM) occurs in 2-3 people per 100,000 [Waters J D, Rose B, Gonda D D et al., “Immediate post-operative brachytherapy prior to irradiation and temozolomide for newly diagnosed glioblastoma,” J. Neurooncol. 113(3):467-477 (2013)]. This relatively rare tumor of the brain has a significant overall mortality due to its refractory response to treatment. The standard of care therapy for GBMs is maximal safe surgical resection, radiation therapy and concurrent chemotherapy with temozolomide (TMZ). However, even with temozolomide therapy, GBMs have a dismal prognosis with a median survival of only 14 months [Id.].

Diffuse intrinsic pontine glioma (DIPG), a life threatening brain cancer in children aged 5-10, is characterized by very low survival rates. The main hurdle in treatment arises because the tumor cells grow in between and around normal cells. It is nearly impossible to surgically remove a tumor in this area because it interferes with the functioning of this vital area of the brain.

One often used chemotherapy for brain cancers, temozolomide (TMZ), is a second-generation imidazotetrazine prodrug that is converted by pH changes in the cytoplasm of cells to the active alkylating agent 5-(3-methyltriazen-1-yl) imidazole-4-carboxamide [Friedman H S, Kerby T, Calvert H, “Temozolomide and treatment of malignant glioma,” Clin. Cancer Res. 6(7):2585-2597 (2000); Patil R, Portilla-Arias J, Ding H et al., “Temozolomide delivery to tumor cells by a multifunctional nano vehicle based on poly(beta-L-malic acid),” Pharm. Res. 27(11):2317-2329 (2010)]. This drug has been unable to achieve a cure for patients with GBMs in spite of activity in mouse models, generally due to inefficient, non-specific delivery.

### Micelle Active Agent Carriers

In view of the these challenges, provided herein are targeted micelle active agent carriers, suitably useful for treating cancers such as brain tumors.

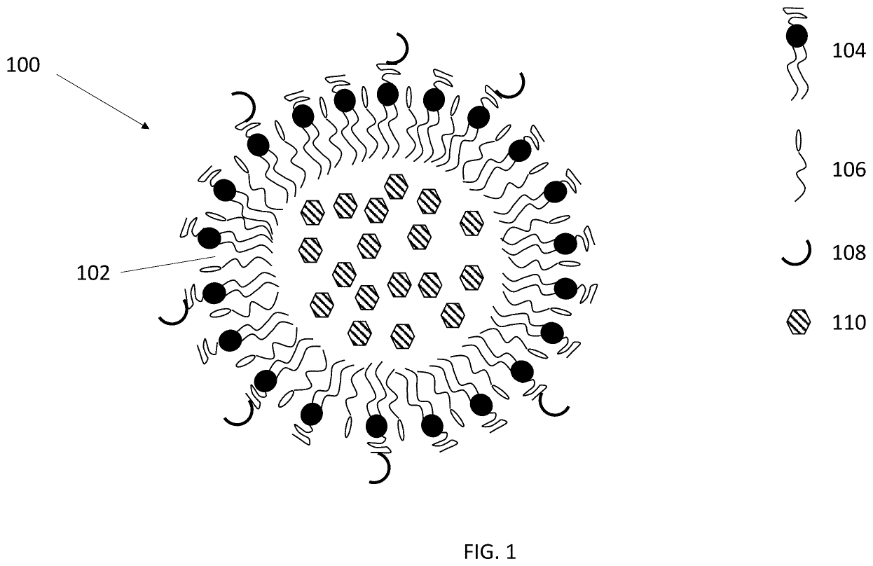

For example, FIG. 1 shows an exemplary targeted micelle active agent carrier 100 (also called “carrier” herein). Targeted micelle active agent carrier 100 suitably includes a micellar structure 102. As used herein “micelle” and “micellar structure” refers to a structure which includes amphipathic molecules, which self assemble into a substantially spherical form in an aqueous solution. Micellar structure 102 suitably includes one or more amphipathic molecules 104, or other micelle forming components, which can include various lipids, polymers (e.g., block co-polymers), etc. Amphipathic molecules, as used herein, refer to molecules which contain both hydrophilic and hydrophilic parts, for example, a hydrophilic head-group and a hydrophobic tail(s). In embodiments, the targeted micelle active agent carrier 100 can include more than one different type of amphipathic molecule 104, for example more than one different type of lipid, such as one or more different phospholipids, such as phosphatidylcholine lipids. The micellar structures can also include additional components, such as cholesterol or other sterols, which can help strengthen the micellar structure, or impart other desired characteristics.

It should be understood that the size, structure and orientation of targeted micelle active agent carrier 100 shown in FIG. 1 is for illustrative purposes only, and is not meant to limit the claimed invention.

Exemplary amphipathic molecules 104 include lipids such as phospholipids, sphingolipids, glycerolipids, etc. Amphipathic molecules 104 can include various carbon chain lengths, e.g., C12-C20, and can be saturated or unsaturated lipid chains, and can contain various headgroups as known in the art. Both di-chain and single chain lipids, or other amphipathic structures, can be used in the formation of micelle structure 102. In embodiments, one or more amphipathic molecules 104 include a poly(ethylene glycol)-lipid or PEG-lipid. Poly(ethylene glycol) is a polymer, which when conjugated to a lipid, provides a steric barrier or “STEALTH” effect to the surface of micelles and liposomes that contain such lipids, allowing for increased circulation, decreased opsonization, and improved retention time in tissues. Suitable molecular weights for the PEG molecules range from about 750 MW to about 5,000 MW, suitably about 1,000-2,000 MW. Exemplary PEG lipids that can be used in the targeted micelle active agent carriers include various PEG-phospholipids, such as PEG-phosphatidylethanolamine-amine (PEG-PE-amine), suitably 1,2-distearoyl-sn-glycero-3-phosphoethanolamine-N-[maleimide(polyethylene glycol)-2000] (DSPE-PEG(2000)), 1,2-diacyl-sn-glycero-3-phosphoethanolamine-N-[aminopoly(ethylene glycol)-2000.

The structure of DSPE-PEG(2000) is shown below for illustrative purposes.

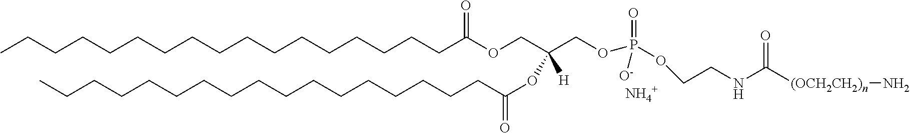

Micellar structure 102 also includes a pH sensitive molecule 106. As used herein, “pH sensitive molecule” refers to a molecule which, upon contact with, or a lowering of the pH surrounding the pH sensitive molecule, to less than about pH 6.0, begins to cause a restructuring of micellar structure 102, thereby allowing for release of the contents entrapped within the micellar structure. Exemplary pH sensitive molecules 106 for use in the targeted micelle active agent carriers described herein, include for example, N-palmitoyl homocysteine (PHC) and D-α-tocopheryl polyethylene glycol succinate (TPGS).

N-palmitoyl homocysteine (PHC) is represented by the following chemical structure:

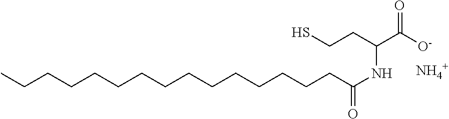 [SMILES: CCCCCCCCCCCCCCCC(=O)NC(CCS)C(=O)[O-] ; [NH4+]]

D-α-tocopheryl polyethylene glycol succinate (TPGS) is represented by the following chemical structure:

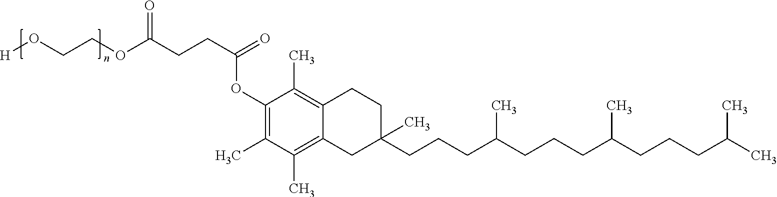

Other pH sensitive molecules include various polymers, which can disrupt a micellar structure upon a lowering of the pH to 6 or less, including for example, poly(methacrylic acid) polymers, poly(vinylpyridine) polymers and poly(vinylimidazole) polymers.

Inclusion of the pH sensitive molecule in the targeted micelle active agent carrier, allows for release of entrapped or encapsulated active agents from the micellar structure, when the targeted micelle active agent carrier is internalized in a cell, for example via an endosomal pathway, and the local pH drops. The reduce pH of some tumors, including the interstitial space, can also be used as a mechanism for triggering or assisting release from the micellar structures.

Also included in the targeted micelle active agent carriers 100 described herein is a targeting moiety 108. As used herein “targeted” when referring to the carriers described herein refers to the use of “targeting moiety” to impart a directed delivery to the carriers. “Targeting moiety” refers to any ligand or suitable molecule that can be associated with a micellar structure, suitably via a PEG-lipid, including for example via chemical conjugation to an amine on the PEG polymer, and provide directed delivery to a cell-surface protein, antibody, tissue, organ, etc., within the body. Exemplary targeting moieties for use in the practice of the present invention include, but are not limited to, proteins, peptides, antibodies, antibody fragments (including Fab′ fragments and single chain Fv fragments) and sugars, as well as other targeting molecules.

In embodiments, targeting moiety 108 targets a receptor tyrosine kinase (RTK) receptor, and is a platelet-derived growth factor (PDGF) peptide or an epidermal growth factor (EGF) peptide. As described herein, it has been surprisingly found that through the use of a PDGF peptide or an EGF peptide, the carriers described herein are able to pass through the blood-brain barrier, and selectively target surface markers on brain tumor cells.

In embodiments of the targeted micelle active agent carriers described herein, targeting moiety 108 and micellar structure 102 are present at a molar ratio of about 0.001 to about 0.02 (targeting moiety:micellar structure). In these ratios, the composition of micellar structure 102 includes the combined molar amounts of all of the lipid/amphiphile components, the pH sensitive molecules, as well as other compounds included in the micellar structure. For example, micellar structure 102 can include PEG-PE amine, hydrogenated soy phosphatidylcholine (HSPC) (or other di- or single-chain phosphatidylcholine lipid), the pH sensitive molecules (e.g., PHC or TPGS) and a sterol, such as cholesterol. In embodiments, the targeting moiety:micellar structure are present at a molar ratio of about 0.005 to about 0.015, about 0.007 to about 0.015, about 0.006 to about 0.01, about 0.007 to about 0.01, about 0.008 to about 0.01, or about 0.005, about 0.006, about 0.007, about 0.008, about 0.0081, about 0.0082, about 0.0083, about 0.0084, about 0.0085, about 0.0086, about 0.0087, about 0.0088, about 0.0089, about 0.0090, about 0.0091, about 0.0092, about 0.0093, about 0.0094, about 0.0095, about 0.0096, about 0.0097, about 0.0098, about 0.0099, about 0.01, about 0.011, about 0.012, about 0.013, about 0.014 or about 0.015 (targeting moiety:micellar structure).

In further embodiments of the targeted micelle active agent carriers described herein, targeting moiety 108 and PEG-PE lipid (e.g., PEG-PE amine) lipid are present at a molar ratio of about 0.04 to about 0.08 (targeting moiety:PEG-PE lipid). For example, in embodiments, the targeting moiety:PEG-PE lipid are present at a molar ratio of about 0.05 to about 0.08, about 0.06 to about 0.07, about 0.065 to about 0.07, or about 0.06, about 0.061, about 0.062, about 0.063, about 0.064, about 0.065, about 0.066, about 0.067, about 0.068, about 0.069, about 0.070, about 0.071, about 0.072, about 0.073, about 0.074, or about 0.075 (targeting moiety:PEG-PE lipid).

In suitable embodiments, the targeted micelle active agent carriers 100 described herein are generally spherical, or nearly spherical, in shape; that is having a relatively uniform cross-sectional diameter. Suitably, the polymeric nanoparticles described herein will have a size (i.e., diameter) of about 1 nm to about 20 nm, more suitably about 5 nm to about 15 nm, about 8 nm to about 15 nm, about 9 nm to about 15 nm, about 9 nm to about 12 nm, or about 5 nm, about 6 nm, about 7 nm, about 8 nm, about 9 nm, about 10 nm, about 11 nm, about 12 nm, about 13 nm, about 14 nm or about 15 nm.

The targeted micelle active agent carriers 100 described herein can include an active agent 110, encapsulated or otherwise associated with micellar structure 102. As the center of micellar structure 102 creates a hydrophobic core or sink, active agents with poor water solubility can suitably be contained within this core. In addition, poorly soluble active agents can also be contained within the hydrocarbon chain region of the components of the micellar structure. In further embodiments, water soluble active agents can be associated with the head-group portion of the micellar structure, being associated or incorporated with this water-soluble portion of the amphipathic molecules.

Exemplary active agents include chemical chemotherapeutics, antineoplastic agents, steroids, antihistaminic agents, neuropharmacologic agents, anti-inflammatory agents, anticoagulants, vasodilators, central nervous system-active agents, anesthetics, anti-inflammatory agents, etc.

In embodiments, active agent 110 is a chemotherapeutic agent, including but not limited to, microtubule interference agents, topoisomerase inhibitors, alkylating agents, thymidylate synthase inhibitors, irreversible steroidal aromatase inactivators, anti-metabolites, pyrimidine antagonists, purine antagonists, ribonucleotide reductase inhibitors, and kinase inhibitors. Microtubule interference agents are those agents which induce disorganized microtubule formation, disrupting mitosis and DNA synthesis and include the taxanes, for example, paclitaxel and docetaxel; vinca alkyloids such as vinblastine, vincristine and vindesine. Topoisomerase inhibitors which act by breaking DNA, include two types, topoisomerase I and topoisomerase II inhibitors. Topoisomerase I inhibitors include but are not limited to irinotecan (CPT-11). Topoisomerase II inhibitors include, e.g., doxorubicin and epirubicin. Other toposiomerase inhibitors useful in the present invention include but are not limited to etopside, teniposide, idarubicin and daunorubicin. Alkylating agents which act by damaging DNA, such as chlorambucil, melphalan, cyclophosphamide, ifosfamide, temozolomide (TMZ), thiotepa, mitomycin C, busulfan, carmustine (BCNU) and lomustine (CCNU) have been shown to be useful chemotherapy agents. The alkylating agents also include the platins such as carboplatin and cisplatin which have been shown to be useful chemotherapy agents, even though they are not alkylators, but rather act by covalently bonding DNA. Thymidylate synthase inhibitors, which interfere with transcription by metabolizing to false bases of DNA and RNA, include, e.g., 5-fluorouracil and capecitabine. Irreversible steroidal aromatase inhibitors, which act as false substrates for the aromatase enzyme, include but are not limited to AROMASIN®. Anti-metabolites such as folate antagonists, methotrexate and trimetrexate have been found to be useful as chemotherapeutic agents. Pyrimidine antagonists such as fluorouracil, fluorodeoxyuridine and azacytidine have been found to be useful as chemotherapeutic agents. Purine antagonists have been found to be useful as chemotherapeutic agents and include agents such as mercaptopurine, thioguanine and pentostatin. Sugar modified analogs also useful as chemotherapeutic agents include cytarabine and fludarabine. Ribonucleotide reductase inhibitors have been found to be useful as chemotherapeutic agents and include agents such as hydroxyurea.

As described herein, temozolomide, which has the following chemical structure, is suitably used in the carriers as active agent 110.

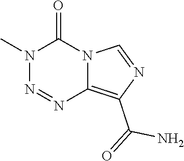

In additional embodiments, provided herein are targeted micelle active agent carriers 100, which include micellar structure 102 comprising poly(ethylene glycol)-phosphatidylethanolamine-amine (PEG-PE-amine) and D-α-tocopheryl polyethylene glycol succinate as pH sensitive molecule 106. Also included is targeting moiety 108 associated with the PEG-PE-amine, and active agent 110 encapsulated or contained within the micellar structure.

In exemplary embodiments, targeting moiety 108 targets a receptor tyrosine kinase (RTK) receptor, and suitably is a platelet-derived growth factor (PDGF) peptide or an epidermal growth factor (EGF) peptide.

Exemplary active agents, including chemotherapeutics, are described herein. In embodiments, active agent 110 is the chemotherapeutic, temozolomide.

As described herein, micellar structure 102 can further include one or more additional lipids or other structures, including for example, a phosphatidylcholine lipid and a sterol, such as cholesterol.

In embodiments, the molar ratio of targeting moiety and the micellar structure are present at about 0.005 to about 0.01 (targeting moiety:micellar structure). As described herein, it has been determined that the ratio of targeting moiety to micellar structure (suitably about 0.065-0.07, or about 0.068) provides an unexpected increase in the ability of the carriers to cross the blood-brain barrier, and to target brain cancer cells for delivery of entrapped or encapsulated active agents within the micellar structure.

The targeted micelle active agent carriers are suitably prepared by a thin-film hydration technique, in which the components of the micelle structure and active agent are co-dissolved in a suitable solvent (e.g., chloroform, DMSO, etc.), dried down into a film, and then hydrated to form micellar structures. Following sonication to produce the desired micelle size, the desired targeting moiety can then be suitably added, by facilitating conjugation to the PEG-PE lipid, for example, via chemical bonding to an amine group on the PEG.

### Methods of Treatment

In further embodiments, provided herein are methods of treating a brain cancer in a patient. In such embodiments, targeted micelle active agent carriers 100 as described herein are administered to a patient. As described throughout, the targeted micelle active agent carriers cross the blood-brain barrier to target the brain cancer and deliver the active agent. It has been determined that the unique structure of the targeted micelle active agent carriers 100, including for example targeting to a receptor tyrosine kinase (RTK) receptor, and the ratios of components described herein, provides enhanced delivery and efficacy for the treatment of brain tumors/cancers.

Methods of administration are well known in the art, and include for example, intravenous administration, oral administration, sublingual administration, intramuscular administration, intralesional administration, intradermal administration, transdermal administration, intraocular administration, intraperitoneal administration, percutaneous administration, aerosol administration, intranasal administration, intraorgan administration, intracereberal administration, topical administration, subcutaneous administration, endoscopic administration, slow release implant, administration via an osmotic or mechanical pump and administration via inhalation.

The carriers made in accordance with the methods of this invention can be provided in the form of kits for use in administration to a patient Suitable kits can comprise, in separate, suitable containers, a lyophilized or freeze-dried form of the carriers described herein. The dried micellar structure can be mixed under sterile conditions with a suitable buffer, including simply sterile water or saline, as well as other buffers, and administered to a patient within a reasonable period of time, generally from about 30 minutes to about 24 hours, after preparation. In additional embodiments, the carriers described herein can be provided in solution form, preferably formulated in sterile water-for-injection, and can include appropriate buffers, osmolarity control agents, etc. These formulations can then be directly administered to a patient via injection, or can be prepared as intravenous drip bags, etc.

The methods of treatment described herein suitably are useful for the treatment of a glioblastoma in a patient. As described herein, the targeted micelle active agent carrier crosses the blood-brain barrier to target the glioblastoma and deliver the active agent. In exemplary embodiments of the methods of treatment, the targeting moiety is a platelet-derived growth factor (PDGF) peptide and the targeting moiety and the micellar structure are present at a molar ratio of about 0.008 to about 0.01 (targeting moiety:micellar structure), more suitably about 0.065 to about 0.07, or about 0.068.

### EXAMPLES

### Example 1: Preparation and Delivery of Targeted Micelle Active Agent Carriers Including PHC

### Methods:

### Materials and Methods

### Synthesis of Micelle-Encapsulated Temozolomide (TMZ)

Dimethyl sulfoxide (DMSO) was added to TMZ and sonicated for 30 min in a water bath at room temperature. The TMZ was then mixed with amino-PEG-PE (1,2-diacyl-sn-glycero-3-phosphoethanolamine-N-[aminopoly(ethylene glycol)]; 870320P; Avanti Polar Lipids, AL, USA) and 0.5 mg of PHC (N-palmitoyl homocysteine [ammonium salt]); 880128P; Avanti Polar Lipids, AL, USA) and suspended in chloroform. The solvent was evaporated in a vacuum oven for 1 h at room temperature. The pellet obtained after evaporation was heated to 80° C. and dissolved in nanopure water (18 mΩ) to produce PEG-amine functionalized micelles. The micelle solution was sonicated for 1 h in a water bath and subsequently filtered using a 0.2 μm syringe filter to remove aggregates. For the synthesis of PDGFR-targeted micelles containing TMZ (PMTMZ), micelle encapsulated TMZ (MTMZ) solution was used for peptide conjugation (1:1 ratio of carboxyl group on peptide to amine group on the micelles at 30% coverage of amines). The PDGF peptide (PDGF pep) sequence was yITLPPPRPFFK (SEQ ID NO:1) (Peptides International, KY, USA). After 15 min of incubation at room temperature, phosphate buffered saline (PBS; pH ˜12) was added to bring the pH back to 7.5. PDGF peptide solution was added to the micelle solution and left incubating for 2 h at room temperature. After 2 h, excess peptide was purified using a 10K MWCO ultracentrifugal device (EMD Millipore, MA, USA) at least three-times at 4000 rpm for 15 min at 4° C. For dye labeling, MTMZ and PMTMZ solution were added to NHS Dylight 680 (ratio of covering 30% amines on the micelles, Thermo Scientific, IL, USA), respectively. PBS buffer (pH 7.2) was added to the solution. The solution was incubated for 1 h at room temperature. After 1 h, excess dye was purified using 10K MWCO ultracentrifugal device three-times.

### Characterization of Micelle-Encapsulated TMZ

The concentrations of MTMZ and PMTMZ were determined by ultraviolet-visible (UV-Vis) absorption using a Biotek microplate spectrophotometer (Winooski, Vt., USA). Dynamic light scattering (DLS) analysis and zeta potential analysis of MTMZ and PMTMZ in aqueous solution was performed on a ZetaPALS particle analyzer (Brookhaven Instruments, NY, USA). PBS (pH 7.2) was used as the starting solvent. The respective aqueous master solution was diluted fivefold and sonicated for 1 h to prevent aggregation. The solution was filtered using a 0.2 μm syringe filter before taking the measurements. Zeta (ξ) potential was automatically calculated from electrophoretic mobility based on the Smoluchowski equation, v=(eE/η)ξ, where v is the measured electrophoretic velocity, η is the viscosity, e is the electrical permittivity of the electrolytic solution and E is the electric field.

Negative-stain transmission electron micrographs (TEM) of MTMZ and PMTMZ were taken by spreading 10 μl of MTMZ or PMTMZ solution (˜1 μM) on a carbon-coated copper grid. Excess solution was removed with filter paper after 10 min, followed by the addition of 10 μl of saturated uranyl acetate solution (2% w/v). After another 10 min, the excess stain was removed with filter paper. The sample was visualized with a JEOL 200CX transmission electron microscope (JEOL, MA, USA) at 80 kV, equipped with a digital camera.

For pH change experiments, PBS buffers of pH 4-10 were prepared. The pH of PBS buffer (pH 7.2) was changed to alternate pHs (pH 4-10) by adding sodium hydroxide or hydrochloric acid to assess the stability of the micelle at various pHs, thereby keeping the salt concentration in the PBS constant. 5 μl of MTMZ or PMTMZ (˜10-4 M) were placed in a 96 well plate. 200 μl of the respective PBS buffers were added to a well. The wells were incubated for 4 h. After 4 h, UV-Vis measurements were recorded at 325 nm (TMZ excitation).

### In Vitro Treatment of Micelle Encapsulated TMZ

Two types of GBM cell lines, U87 and LN229 (ATCC, VA, USA), were used. U87 is a primary human GBM cell line with an epithelial morphology which was acquired from a stage IV 44-year-old cancer patient [Clark M J, Homer N, O'Connor B D et al., “U87MG decoded: the genomic sequence of a cytogenetically aberrant human cancer cell line,” PLoS Genet. 6(1):e1000832 (2010).]. LN229 is another human GBM cell line derived from brain/right frontal parieto-occipital cortex of a 60-year old female GBM patient with similar epithelial morphology [Ishii N, Maier D, Merlo A et al., “Frequent co-alterations of TP53, p16/CDKN2A, p14ARF, PTEN tumor suppressor genes in human glioma cell lines,” Brain Pathol. 9(3):469-479 (1999).]. LN229 or U87 cells were plated on a 25×25 mm coverslip at a density of 30,000 cells per coverslip and maintained overnight in cDMEM at 37° C. in an incubator supplied with 5% CO₂. Twenty-four hours after plating, cells were treated with increasing TMZ concentrations of MTMZ and PMTMZ with a final volume of 300 μl for a total of 4 h. Immunostaining was done to observe the co-localization of the drug and receptors. After treatment, the cells were washed with media and then fixed with 8% paraformaldehyde for 10 min followed by three washes with PBS buffer. The fixed cells were blocked with 3% goat serum for 1 h. Following blocking, the cells were incubated with primary anti-PDGFR (1:500; sc-432; Santa Cruz Biotech, TX, USA) for 2 h. The cells were washed with PBS buffer followed by incubation with secondary goat antirabbit Alexa 488 antibody (1:1000; A11034; Life Technologies, NY, USA). For staining of nuclei, cells were incubated with DAPI (4′,6-diamidino-2-phenylindole) (1:7500). The uptake and co-localization of particles was visualized by a fluorescence microscope using a Leica DM 4000B microscope (Leica Microsystems, IL, USA). The images were analyzed using ImageJ (NIH) software for relative normalized intensities for comparison analysis. The prior experimental protocol was then replicated in a longitudinal study using MTMZ and PMTMZ at concentrations of 0.504 or 1 μM. Cells plated on coverslips were incubated with either MTMZ or PMTMZ over increasing time. Coverslips were fixed with paraformaldehyde at 5, 15, 30 and 60 min after incubation for short-term observation and at 4, 8, 16 and 24 h for long-term examination. The uptake and co-localization of particles was visualized by fluorescence microscope using a Leica DM 4000B microscope (Leica Microsystems). The images were analyzed using ImageJ (NIH) software for relative normalized intensities for comparison analysis.

### Cytotoxicity of TMZ Micelles

Three cytotoxicity experiments, using Guava Via-Count Assay and flow cytometry (EMD Millipore, MA, USA), were performed using increasing concentrations of free TMZ, MTMZ and PMTMZ over increasing time. Cells (30,000 cells per well) were plated in 24-well plates and incubated overnight at 37° C. with 5% CO₂. A TMZ solution (1 mg ml⁻¹) in DMSO was prepared and stored at 4° C. The stock solution was diluted with alpha-MEM and used to prepare free TMZ at increasing concentrations of 0-100 04. Independently, both 1 μM and 10 04 samples of PMTMZ and MTMZ were prepared with alpha-MEM. The triplicate wells were treated with concentrations at a constant volume of 200 μl per well. After 24 h, cell viability was performed using Guava ViaCount Assay and flow cytometry (EMD Millipore). The remaining wells were retreated with the appropriate concentrations over a 10 day period. Viability data was collected and analyzed.

### Inhibition of Receptor Cycling Using Brefeldin a In Vitro

U87 cells were plated on 25×25 mm coverslips at a density of 30,000 cells per coverslip and maintained overnight in media at 37° C. in an incubator supplied with 5% CO₂. Twenty four hours after plating, one set of cells was treated with 250 μl of brefeldin A (BA) solution (10 μg ml-1 in media) and incubated for 1 h (+BA). Another set of coverslips was left with 250 μl of media as −BA controls. For the +BA set of cells, the BA solution in media was replaced with 250 μl of 500 nM MTMZ or PMTMZ solutions. The −BA cells were treated with 250 μl of 500 nM MTMZ or PMTMZ solutions. Both set of cells were incubated with the micelles for 0.5, 1, 4 and 6 h, respectively. After treatment, the cells were washed with media and then fixed with 4% paraformaldehyde for 10 min followed by three washes with PBS buffer. For staining of nuclei, cells were incubated with DAPI (1:7500). Uptake and co-localization of micelles were visualized by fluorescence microscope using a Leica DM 4000B microscope (Leica Microsystems, IL, USA). The images were analyzed using ImageJ software for relative normalized intensities for comparison analysis.

### Orthotopic Tumor Implantation

For orthotopic brain tumor implants, athymic nude mice (NCR Nu;Nu; Charles River Laboratory, MD, USA) were anesthetized by intraperitoneal injection of 50 mg kg⁻¹ bodyweight ketamine/xylazine and fitted into a stereotaxic rodent frame (David Kopf Instruments, CA, USA). A small incision was made just lateral left to midline to expose the bregma suture. A small (1.0 mm) burr hole was drilled at AP=+1, ML=−2.5 from bregma. Glioblastoma cells (U87, 300,000 cells in 3 μl) were slowly deposited at a rate of 1 μl per minute in the left striatum at a depth of −3 mm from dura with a 10 μl Hamilton syringe (26G blunt needle, Fisher Scientific, PA, USA). The needle was slowly withdrawn and the incision was closed with 2-3 sutures. The tumors developed for 9 days prior to tail vein injection. Tumor burden and location was evaluated using luciferase activity. At 9 days, luciferin (150 μg ml-1; substrate for luciferase) was injected within the peritoneal cavity. Luminescence measurements were taken using an IVIS 200 imager (PerkinElmer, MA, USA). Animals were fed exclusively on a special rodent diet (Tekland 2018S; Harlan Laboratories, Inc., IN, USA) to reduce autofluorescence. Animal experiments were performed according to policies and guidelines of the Institutional Animal Care and Use Committee (IACUC) at Medical University of South Carolina under approved protocols.

### In Vivo Fluorescence Imaging

Mice with orthotopic tumors were anesthetized with isoflurane and injected intravenously via the tail with either PMTMZ or MTMZ at a dosage of 0.001 mg kg⁻¹ of TMZ per total mouse body weight. Mice were imaged at 0, 1, 4, 6 and 24 h. After live imaging, the mice were euthanized and excised organs were imaged after necropsy. Fluorescent multispectral images were obtained using the Maestro In Vivo Imaging System (PerkinElmer, MA, USA). Multispectral in vivo images were acquired under a constant exposure of 2000 ms with an orange filter acquisition setting of 630-850 nm in 2 nm increments. Multispectral images were unmixed into their component spectra (Dylight 680, autofluorescence, and background) and these component images were used to gain quantitative information in terms of average fluorescence intensity by creating regions of interest (ROIs) around the organs in the Dylight 680 component images.

### Results:

Micelles composed of PEG-PE amine (1,2-distearoyl-sn-glycero-3-phosphoethanolamine-N-[amino(poly ethylene glycol)-2000 and PHC (N-palmitoyl homocysteine (ammonium salt)) were prepared to encapsulate hydrophobic TMZ. PHC, a pH sensitive lipid was used to assist in the micelle rupture at acidic pH to ensure the delivery of the cargo inside the micelle core. Amine functionality on PEG-PE amine was utilized for further tailoring of the micelle with targeting peptides (PDGF, yITLPPPRPFFK) (SEQ ID NO:1) containing a carboxyl group and labeling with fluorescent dyes (Dylight 680) for tracking the micelle in in vitro cellular uptake studies.

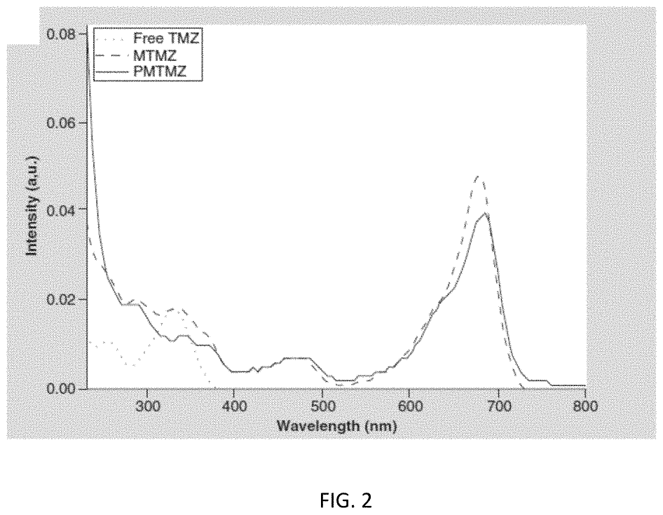

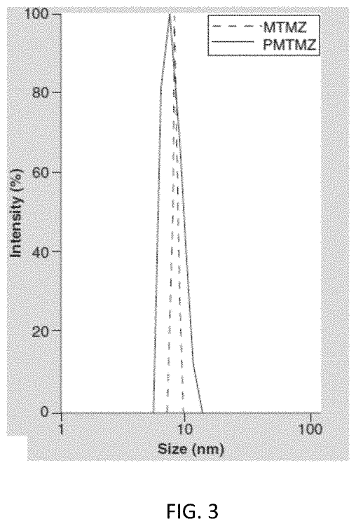

Untargeted, micelle-encapsulated TMZ (MTMZ) and PDGFR-targeted TMZ (PMTMZ) were characterized by DLS, UV-Vis spectroscopy and micelle integrity in physiological buffer. The UV-Vis spectra of MTMZ and PMTMZ showed peaks from TMZ (325 nm) and the fluorophore (680 nm) demonstrating the presence of hydrophobic TMZ inside the core and the fluorescent label on the exterior of the micelles (FIG. 2). DLS data showed both MTMZ and PMTMZ have an average hydrodynamic diameter of around 10±1.2 and 12±2.3 nm, respectively, with a polydispersity index of 0.1 and 0.2% (FIG. 3). The size distribution is determined by the polydispersity index. The lower the value is, the narrower the size distribution or the more uniform the nanoparticle sample. However, attachment of PDGF increased the polydispersity index due to the steric hindrance caused by the cyclic structure of PDGF. The DLS size distribution is identical to the instrumental response function corresponding to a monodispersed sample, indicating that aggregation is negligible. Zeta potential is an indicator of surface charge, which determines particle stability in dispersion. Zeta potentials of MTMZ and PMTMZ were −40.35±4.46 and −45.18±3.71 mV, respectively, as shown in Table 1.

TABLE 1
Characteristics of micelle-encapsulated temozolomide and
PDGF-micelles containing temozolomide nanoparticles.

| | Sample | Sample |
|---|---|---|
| Nanoparticle properties | group: MTMZ | group: PMTMZ |
| Size ± SD (nm) | 10 ± 1.2 | 12 ± 2.3 |
| Polydispersity (%) | 0.1 | 0.2 |
| Zeta potential ± SD (mV) | −40.35 ± 4.47 | −45.19 ± 3.71 |

The micelles were found to be stable in the dispersion state, possessing high absolute values of zeta potential and having negative surface charges. Particle surface conjugation slightly increased the absolute value of the zeta potential.

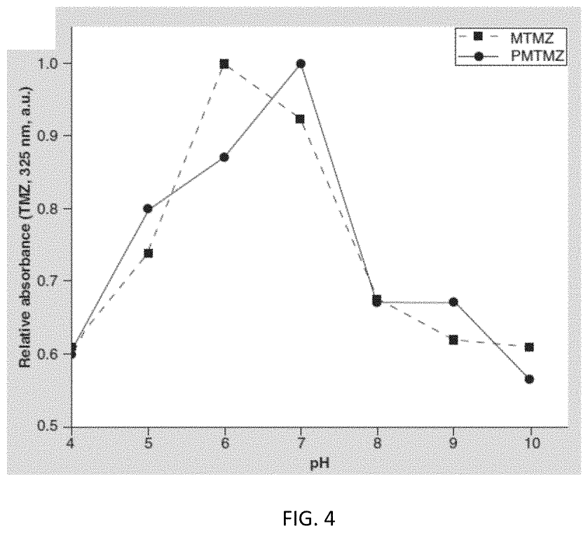

Stability and rupture efficiency of the micelles were evaluated using a pH change assay (FIG. 4). The pH change studies evaluated the range at which the micelles rupture. The micelles were designed to rupture at an endosomal of pH approximately 5.5 to deliver the encapsulated TMZ cargo. These studies illustrated that for both MTMZ and PMTMZ, increased absorbance intensity at 325 nm was seen between pH 6 and 7, indicating that the micelles were intact, holding the hydrophobic TMZ inside its core. Upon decreasing the pH from 7 to 4 (acidic milieu), the intensity was reduced by approximately 34% for MTMZ and 40% for PMTMZ. Upon increasing the pH from 7 to 9 (basic milieu), the intensity declined by approximately 33% for both MTMZ and PMTMZ. This depletion of intensity is attributed to the loss of micelle membrane integrity, which is due to the pH-responsive lipid composition to both the increasing and decreasing pH. TMZ was able to leach out of the micelles and then aggregate within the aqueous solution. TMZ was removed from the optical path of the excitation wavelength. This demonstrates the functional capability of the micelles to release the TMZ at an acidic pH representative of endosomal pH.

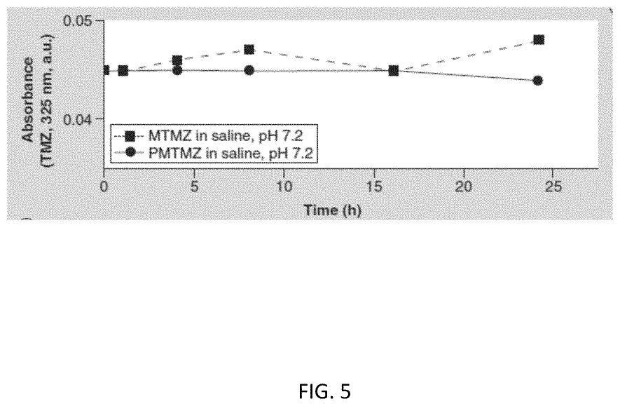

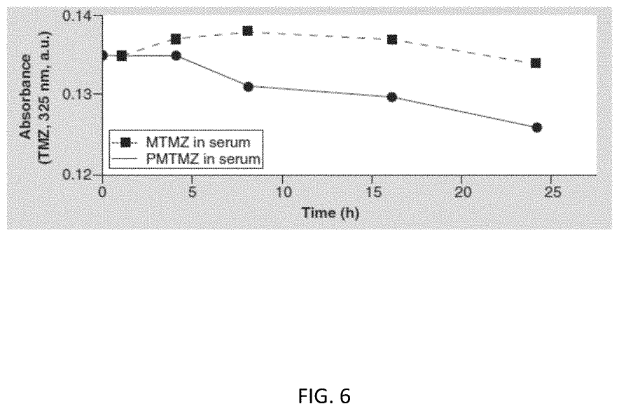

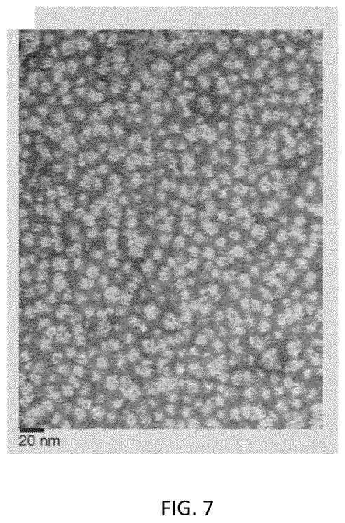

Stability of the MTMZ and PMTMZ was assessed over a 24 h period. To mimic the physiologic environment, the micelles were suspended in saline (PBS, pH 7.2) and absorbance of the drug was examined (FIG. 5). Both carriers were relatively stable over the 24 h since the change in absorbance of the drug was negligible. The slight increase in absorbance for MTMZ can be attributed to instrumental error. In addition, the stability of these micelles was also evaluated in serum since the presence of lipids, amino acids and proteins in the serum can cause micelle instability (FIG. 6). The micelles were slightly less stable than those suspended in saline over the same period with overall loss of absorbance at 325 nm of approximately 5-7%. These micelle stability experiments established the robust nature of the micelles for use in in vivo studies. The self-assembly of the lipids and structural integrity of the micelles were examined using electron microscopy (FIG. 7). TEM revealed the presence of spherical micelles for PMTMZ with a diameter of approximately 12-13 nm.

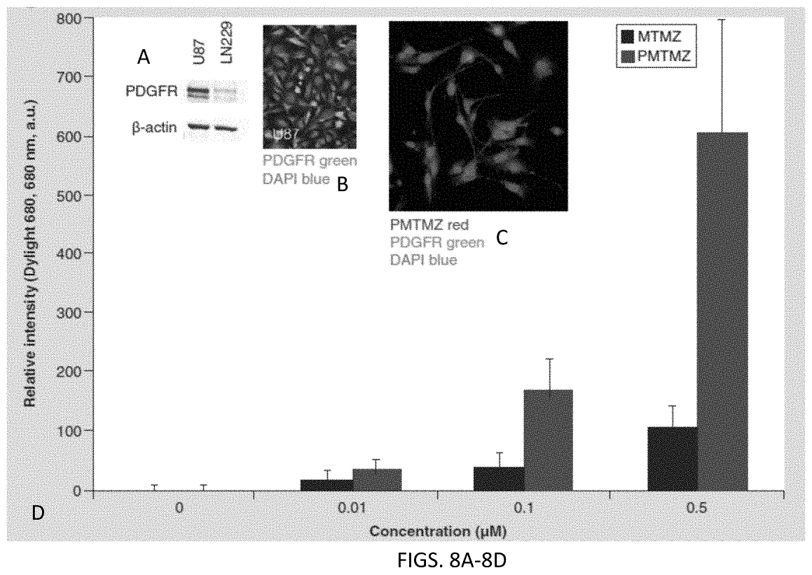

Micelles were functionalized with a PDGF peptide (PDGFpep) to target the PDGFR expressed on the glioma cell surfaces to facilitate targeting and cellular uptake. Theoretical calculations predict that there are 242 PDGF peptides per targeted micelle. Calculations were made as follows: assuming the micelle is a sphere of 10 nm, the surface area (SA) of the sphere was calculated first. Then the total number of lipid molecules in one micelle was calculated by dividing the total SA by the SA of the lipid molecules. Using the molar ratio of the lipids used, the number of PEGPE amine molecules was calculated, which is equivalent to number of PDGF peptides (assuming 100% coupling). Accumulation of the micelles after targeting with the PDGFpep was assessed in vitro utilizing immunofluorescence (FIGS. 8A-8D). U87 cells that overexpress the PDGFR (FIG. 8A) were treated with increasing concentrations of either PMTMZ (targeted) or MTMZ (untargeted) for 4 h at 37° C. PMTMZ was internalized in PDGFR-expressing U87 cells with as little as 0.01 μM micelles, indicating the threshold concentration for uptake (FIG. 8D). At 0.5 μM, significant uptake was observed suggesting receptor-mediated endocytosis. The uptake was quantified and the relative intensity graph demonstrated that PMTMZ uptake is threefold higher than that of MTMZ at 0.1 μM and six fold higher at 0.5 μM. The targeted micelles co-localized with the receptor with a Pearson's correlation coefficient of 0.83 (FIG. 8C). In contrast, few untargeted micelles were taken up during treatment with increasing concentrations and did not co-localize with the receptors (Pearson's correlation coefficient=0.32).

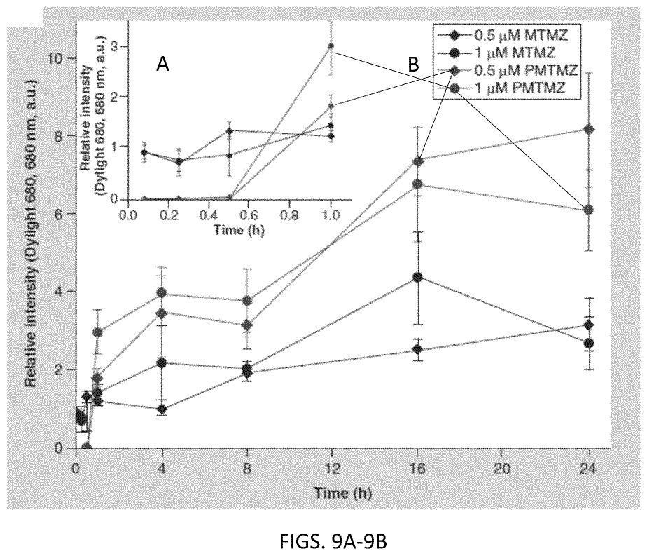

A longitudinal study was performed to demonstrate uptake of PMTMZ and MTMZ over a total period of 24 h (FIGS. 9A-9B). Overall, PMTMZ uptake for 0.5 μM or 1 μM was consistently higher than MTMZ at every time point (1, 4, 8, 16, 24 h). A significant increase was first observed within 30 min to 1 h after treatment was initiated (FIG. 9A). After 24 h, PMTMZ uptake at 0.5 μM was significantly higher (166%) as compared with MTMZ uptake.

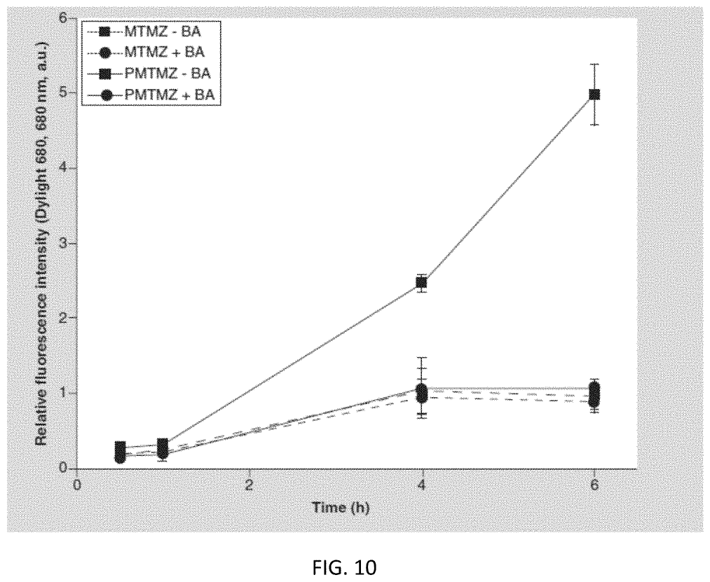

To demonstrate that uptake of PMTMZ was predominantly due to endocytosis associated with the PDGF peptide and not diffusion of the micelles, U87 were treated with brefeldin A (BA), a fungal metabolite that reversibly interferes with intracellular transport and receptor cycling, and examined for uptake (FIG. 10). BA acts by inducing major structural changes in the morphology of endosomes, the trans-Golgi network, and lysosomes by causing the formation of an extensive tubular network and preventing new endosome formation. As noted above, significant fluorescence was observed when U87 were incubated with PMTMZ (−BA, 15.8% increase) over a 6 h period. Fluorescence intensity increased by only 5.06% when U87 were treated with MTMZ (−BA). Preincubation with BA (+BA) decreased the relative fluorescence intensity of PMTMZ incubated cells by 78.2% over time. MTMZ uptake was inhibited to a much lesser extent with BA (8.2%).

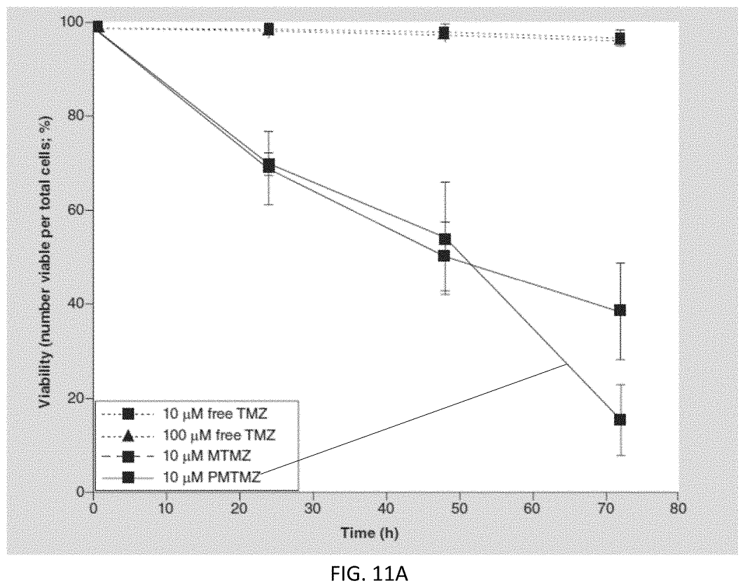

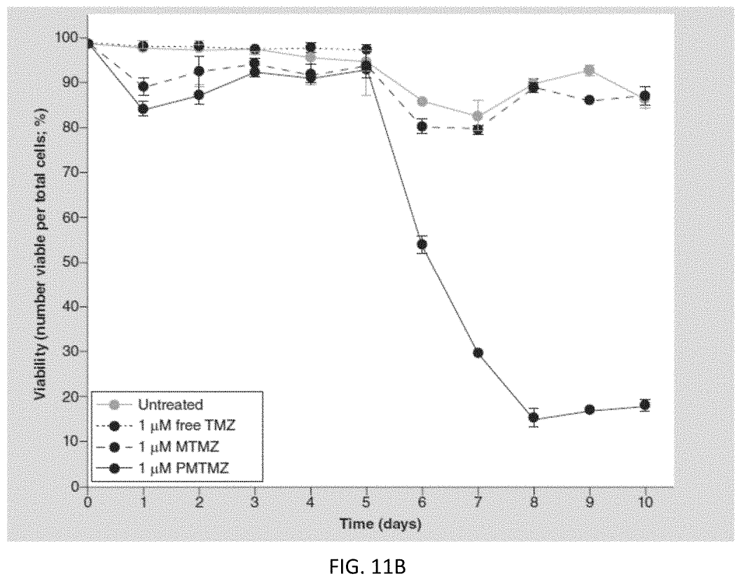

PMTMZ was first evaluated for cell killing efficacy using a short-term cell viability assay (FIG. 11A). Glioma cells (U87) were treated with PMTMZ or MTMZ (10 μM each) or free TMZ (10 or 100 μM each) over the course of 24-72 h with treatments added to fresh media once a day. PMTMZ results were compared directly to either free TMZ or MTMZ measurements. After only 24 h, both PMTMZ and MTMZ began to exhibit more killing (˜30%) than that of equal or increased concentrations of free TMZ. By 72 h, there was a significant difference in the killing between treatment groups; with PMTMZ killing approximately 84% more cells than 10 or 100 μM of free TMZ; and MTMZ killing approximately 61% more cells than either treatment concentration of free TMZ. Overall, between 24 and 72 h, PMTMZ exhibited compelling cell death of approximately 78% with MTMZ following at compelling 44%. In comparison, free TMZ (10 or 100 μM) only illustrated a cell death of compelling 2%.

PMTMZ was then evaluated for cell killing efficacy using a longitudinal cell viability assay with a tenfold decrease in TMZ concentration (FIG. 11B). Glioma cells (U87) were treated with PMTMZ, MTMZ or free TMZ (1 μM each) over a 10 days period with treatments added to fresh media once a day. PMTMZ results were compared directly to either free TMZ or MTMZ measurements. Between 1 and 5 days, there was no significant difference between the treatment groups; free TMZ killed approximately 4% of the cells and PMTMZ and MTMZ had little to no effect on cell death. However, after day 5, PMTMZ dramatically killed the cells at 1 mM (˜82% by day 8). In contrast, MTMZ after day 5 showed no appreciable cell death, maintaining a 1-2% death rate comparable to that of untreated cells.

For FIG. 11A the concentration is tenfold higher for MTMZ and PMTMZ administered to the cells than that of FIG. 11B. It was expected that a decrease in administered concentration would take longer (5 days) to show efficacy as compared with that of a higher concentration over a shorter period of time (3 days). The data show a consistent decrease in cell viability over 3 days at 10 μM PMTMZ (FIG. 11A) and after 5 days 1 μM PMTMZ (FIG. 11B). It appears that untargeted, MTMZ is unable to deliver a significantly toxic dose of TMZ to the cells when only 10 μM is administered.

Mice containing orthotopic gliomas from implanted luciferase expressing U87 cells were first evaluated for tumor burden using in vivo bioluminescence imaging. Luciferase expressing glioma cells were used in conjunction with luciferin substrate (150 μg ml⁻¹) in order to confirm the presence of tumor in the brain and verify the location of the tumor. A standard curve for luciferase activity was generated using increasing cell numbers of U87 (without luciferase expression as control) and U87-luciferase cells incubated with luciferin. U87-luciferase expressing cells showed a linear increase in luminescence with increasing cell numbers. Tumor burden in vivo was approximated to cell number using the standard in vitro curve. After 7 days of growth, tumors contained approximately 12.3 million cells.

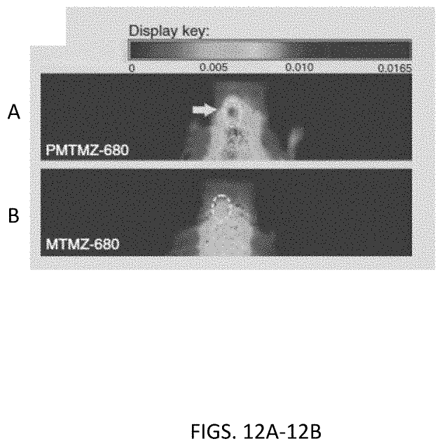

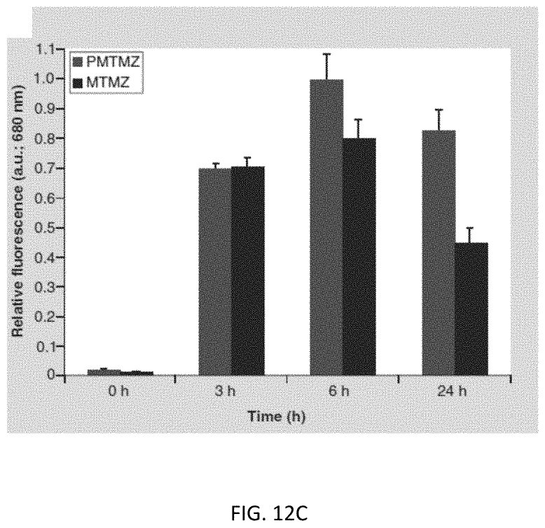

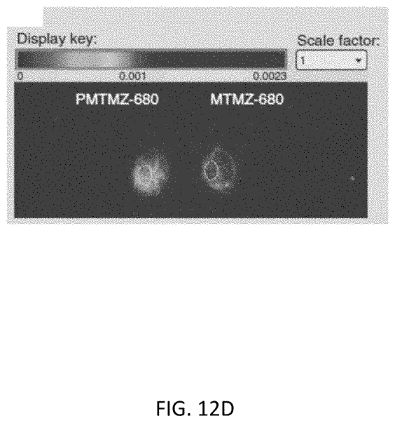

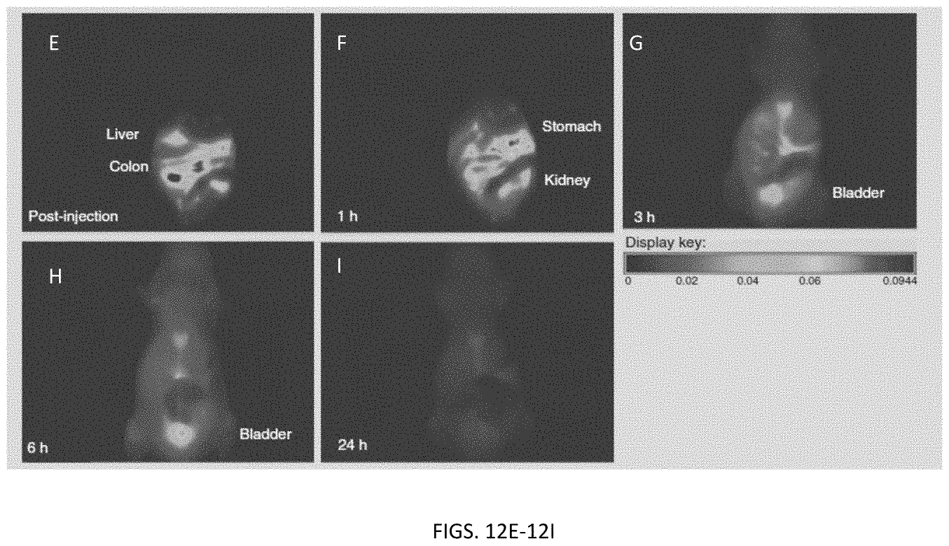

Mice treated with PMTMZ accumulate the nanocarrier in the brain over a 24 h period (FIG. 12A) as compared with those animals treated with untargeted MTMZ (FIG. 12B). Multiple controls were conducted, including mice sham-implanted with PBS instead of cells and mice orthotopically implanted but administered PBS instead of either MTMZ or PMTMZ. No fluorescence was observed in these control mice as compared with the experimental PMTMZ and MTMZ administered mice. A ROI modeled around the craniums of the mice showed significant fluorescence associated with PMTMZ treated animals.

Quantitation of fluorescence intensity in an ROI created around the brain tumor (dashed circle) confirmed the trend observed in the whole animals (FIG. 12C). During short incubation periods (3-6 h), both targeted PMTMZ and untargeted MTMZ micelles were found in the brain tumor. However, after 24 h the untargeted micelles washed away and the tumors retained 40% more of the targeted micelles containing TMZ. To verify that the fluorescence from PMTMZ was attributable to uptake specifically within the brain, the brains of mice injected with either PMTMZ (left) or MTMZ (right) were then excised from euthanized mice and imaged (FIG. 12D). Biodistribution of PMTMZ was also observed using real time in vivo fluorescence (FIGS. 12E-12I). Animals were imaged in the ventral position during the 24-h period. Since this is topographic fluorescence imaging, the 3 mm depth of the tumor in the dorsal striatum of the brain is not seen from the ventral side because of tissue and bone scattering. PMTMZ was found quickly in the colon just postinjection and declined rapidly over 6 h. PMTMZ then was excreted through the urinary bladder until almost all fluorescence washed away after 24 h. No difference was observed in the excretion pattern of MTMZ as compared with PMTMZ.

### Discussion

Temozolomide is an effective, US FDA-approved chemotherapeutic known for its comprehensive antitumor activity in tumor models, and it is the current standard of care for glioblastoma multiforme. In previous studies TMZ has proven potent in in vivo systems by traversing the CNS, demonstrating accumulation in malignant tissues. Despite its exceptional tumor regression activity, TMZ is extremely hydrophobic, thereby reducing its bioavailability. In addition, hydrophobicity hampers its ability to cross the blood-brain barrier (BBB), which remains a considerable obstacle to glioma therapy. This necessitates formulation of a drug delivery system which can encompass these requirements: a tailored surface on the carrier to attach biomolecules for targeted drug delivery; a biocompatible coating which can efficiently encapsulate the hydrophobic drug thereby reducing cytotoxicity; and stimuli-induced (i.e., pH) disruption of the carrier agent for drug release to the desired environment.

Micelles are the preferred choice of nanocarrier in comparison to other potential carriers based on their composition. Micelles are composed of amphiphilic lipid molecules with a hydrophobic core and hydrophilic exterior. The hydrophobic core of the micelles serves as a container for weakly water-soluble drugs while the outer shell can protect encapsulated drugs and prevent the drugs from leaching out. Recently, polymeric micelles have been utilized as drug carriers due to their properties of hydrophilicity and degradability and due to the ability to tailor their exterior surface with multiple functionalities to attach various biomolecules. In addition, PEG (hydrophilic polymer) can be incorporated in the micellar composition in order to block nonspecific interaction and prolong the blood circulation times of the micelles in a biological milieu. Other hydrophobic drugs like lomustine, carmustine and 5-fluorouracil have been encapsulated inside micelles composed of poly(propylene oxide) (PPO), poly(D, L-lactic acid) (PDLLA), poly(ecaprolactone) (PCL), poly(L-asparate) and poloxamers against brain tumors. The present system is distinct because it incorporates a pH-sensitive molecule (PHC), a targeting ligand (PDGF), and PEG for prolonged circulation, making it a multifunctional micelle within the tumor environment. Since MTMZ and PMTMZ range between 10 and 15 nm, their size (<100 nm) is advantageous for these carriers to cross the BBB, which prohibits larger nanocarriers. The main mechanisms by which micelles target brain tumors are passive diffusion through a disrupted BBB via permeability and enhanced permeability and retention (EPR) effect to reach glioma cells or active receptor-mediated endocytosis to the tumor region.

Treatment of GBMs has been limited by a number of medical obstacles, not least of which is the challenge of achieving adequate chemotherapeutic concentrations in the tumor without systemic toxicities. As discussed above, micelle-encapsulated therapies address this obstacle.

Although PDGFR is expressed at low to moderate levels in other organs, focal amplification of the PDGFR gene and overexpression of PDGFR is frequently observed in aggressive brain tumors. The PDGFR targeting induces the GBM tumor cells to internalize the micelles via receptor-mediated endocytosis. These internalized micelles are then within an appropriate pH environment for the intracellular release of TMZ. This approach increases the accumulation of micelles in the relevant regions of the brain (specifically, in the tumor tissue) in order to increase the release of TMZ, which leads to an elevated concentration of TMZ in the tumor itself. Simultaneously, this approach also potentially reduces the risk of systemic toxicity as the micelles are targeted to the GBM, such that lysis of the micelle should preferentially occur in the tumor rather than systemically. The pattern of fluorescence observed in the biodistribution study suggests that the micelles are processed through both hepatobiliary and urinary excretory paths with over 80% clearance from initial excretory organ uptake within 72 h.

Due to the high concentration of TMZ (˜100 μM) utilized in the clinical setting, this concept of an effective and efficient targeting moiety (PMTMZ) is vital. Through the use of the targeted, pH-responsive chemotherapeutic, the dosage can be reduced from approximately 100 μM of free TMZ to 1 μM PMTMZ resulting in more than double the efficacy of glioma cell death and diminished overall systemic effects.

### Conclusion

Targeted micelles loaded with TMZ were designed to increase the delivery of the drug into the brain. TMZ packaged pH-responsive micelles composed of PEG-PE-amine and PHC surface functionalized with PDGF peptide and Dylight 680 fluorophore (PMTMZ) have specific uptake and increased cell killing in glial cells compared with untargeted micelles (MTMZ). In vivo studies demonstrated selective and increased accumulation of PMTMZ in orthotopic gliomas implanted in mice. This study validates the use of a pH-responsive, receptor-mediated targeting moiety for the effective delivery of chemotherapeutics in the treatment of GBM.

Through its ability to overcome the widespread clinical obstacle of crossing the BBB, in addition to significantly decreasing overall systemic toxicity, this hydrophobic drug-loaded carrier creates potential for the selective delivery of other anticancer agents.

### Example 2: RTK Peptide Targeted Micelles Encapsulating TMZ Containing TPGS

### Introduction

Micelles composed of PEG-PE amine (1, 2-distearoyl-sn-glycero-3-phosphoethanolamine-N-[amino(poly ethylene glycol)-2000]) and TPGS (D-α-tocopheryl polyethylene glycol succinate) are synthesized. Amine functionality on PEG-PE amine is utilized for tailoring with PDGF peptide (or other RTK receptors) for targeting RTK receptors on the cell surface and for labeling of the micelles with fluorescent dyes (Dylight 680 or Dylight 755) for tracking the micelle in in vitro cellular uptake assays and in in vivo studies. TPGS, a pH sensitive molecule is utilized to assist in the micelle rupture at acidic pH to ensure the delivery of the cargo inside the micelle core to the cells.

### Synthesis and Characterization Protocol:

### Synthesis of Micelle-Encapsulated Temozolomide (TMZ)

Micelles are designed using the following w/w ratio of 1:1 of the individual components (PEG-PE amine and TPGS). A traditional lipid film hydration method is used to prepare these micelles loaded with TMZ (temozolomide). Typically, 0.5 mg of TMZ is mixed with 50 μl of DMSO at room temperature. After TMZ is dissolved in DMSO, 2 mg of the respective micelle components are dissolved in chloroform (2 ml). The solution is sonicated in an incubator at room temperature for 30 minutes. This solution is evaporated to dryness in a vacuum oven overnight until a dry film is obtained. The dry film is heated to 70° C. and 1 ml of PBS buffer (pH-7.2) at 37° C. is added to the film to form micelles. This micellar solution is sonicated for 1 hour in an incubator water bath at 37° C. This micellar solution is filtered thru a 0.22 μm syringe filter to purify large aggregates to obtain an optically clear solution, size resolved solution of micelles. The solution is stored at 4° C. until further use.

### Conjugation of PDGF Peptide to TMZ Micelles

The TMZ micelle solution obtained above is concentrated to 100 μl via centrifugation with 10 K MWCO ultracentrifugal filter for 15 minutes at room temperature. A 1:1 ratio of carboxyl group on the peptide to amine group on the micelles at 30% coverage of amines on the micelles corresponds to 20 ul of PDGF (1 mg/200 μl in DMSO). EDC (4 μl) and sulfo-NHS (11 μl) in 100 μl of MES buffer (pH 4.5, 10 mg/100 μl) are added to 20 μl of PDGF peptide in 1 ml of MES buffer. After 15 minutes of incubation at room temperature, 700 μl of PBS (pH ˜12) is added to equilibrate the solution (pH 7.5). The micelle solution (200 μl) is added to the peptide solution and incubated for 2 hours at room temperature. After 2 hours, the excess peptides are removed using a 10K MWCO ultracentrifugal device at least 3 times at 4,000 rpm for 15 minutes at 4° C.

### Conjugation of TMZ Micelle-Peptides to NHS Dylight 680

The micelle-peptide solution (200 μl) is added to 1 μl of NHS Dylight 680 (1 mg/200 ul in DMSO at a ratio of covering 30% of the remaining amines). PBS buffer (300 ul, pH 7.4) is added to the solution. The solution is the stirred for 1 hour at room temperature. After 1 hour, excess dye is removed using 10K MWCO ultracentrifugal devices at least 3 times at 4,000 rpm for 15 minutes at 4° C.

### Characterization

Dynamic Light Scattering (DLS) of untargeted and targeted TMZ micelles in aqueous solution is performed to estimate the hydrodynamic diameter. The respective aqueous master solution is diluted five-fold and sonicated for 1 hour to prevent aggregation. The solution is filtered using a 0.2 μm syringe filter before taking the measurements. UV-Vis measurements are conducted to confirm the attachment of respective fluorescent dye conjugated to the labeled micelles.

### Results:

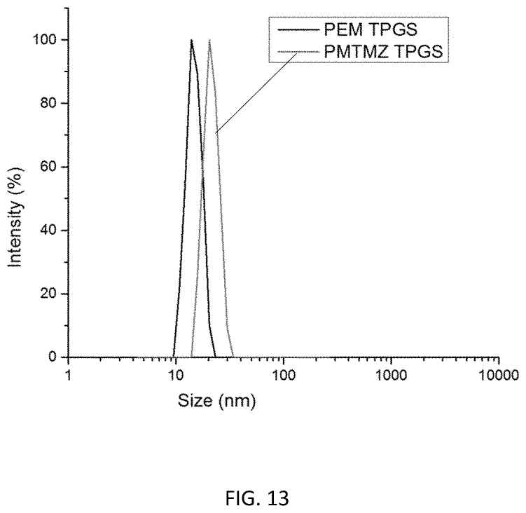

Results of the dynamic light scattering are shown in FIG. 13, illustrating intensity vs. size for micelles of PEG-PE-amine and TPGS, as well as targeted active agent containing micelles, containing TMZ and targeted with the PDGF peptide. The results show that both untargeted (PEM TPGS) and targeted TMZ (PMTMZ) micelles are relatively monodisperse with around 10-20 nm in diameter. The size of the targeted micelles is slightly larger, around 20 nm, possibly due to steric hindrance by PDGF peptide.

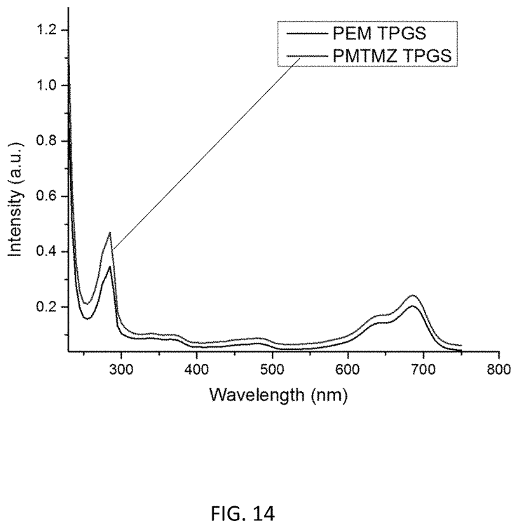

FIG. 14 shows the absorbance spectra of both the micelle preparations. The concentrations of PEM TPGS and PMTMZ TPGS were determined by UV-Vis absorption using a Biotek microplate spectrophotometer. The concentration of TMZ was determined using UV-Vis spectroscopy at 325 nm while the fluorescent dye attachment is confirmed by the peak at 680 nm.

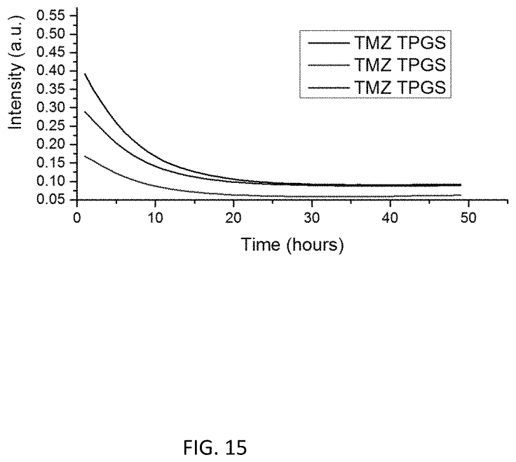

FIG. 15 shows a stability study of PMTMZ TPGS in PBS buffer (pH-7.2) to mimic the biological environment over a period of 48 hours. Absorbance is measured at 325 nm at 1 hour intervals. The experiment is performed in triplicate, and demonstrates that the nanoparticles are stable for 10-24 hours.

It will be readily apparent to one of ordinary skill in the relevant arts that other suitable modifications and adaptations to the methods and applications described herein can be made without departing from the scope of any of the embodiments.

It is to be understood that while certain embodiments have been illustrated and described herein, the claims are not to be limited to the specific forms or arrangement of parts described and shown. In the specification, there have been disclosed illustrative embodiments and, although specific terms are employed, they are used in a generic and descriptive sense only and not for purposes of limitation. Modifications and variations of the embodiments are possible in light of the above teachings. It is therefore to be understood that the embodiments may be practiced otherwise than as specifically described.

All publications, patents and patent applications mentioned in this specification are herein incorporated by reference to the same extent as if each individual publication, patent or patent application was specifically and individually indicated to be incorporated by reference.

1. A targeted micelle active agent carrier, comprising:
a. a micellar structure comprising a poly(ethylene glycol)-lipid (PEG-lipid) and a pH sensitive molecule;
b. a targeting moiety associated with the PEG-lipid; and
c. an active agent encapsulated within the micellar structure.

2. The targeted micelle active agent carrier of claim 1, wherein the PEG-lipid is PEG-phosphatidylethanolamine-amine (PEG-PE-amine).

3. The targeted micelle active agent carrier of claim 1, wherein the pH sensitive molecule is N-palmitoyl homocysteine (PHC) or D-α-tocopheryl polyethylene glycol succinate (TPGS).

4. The targeted micelle active agent carrier of claim 1, wherein the targeting moiety targets a receptor tyrosine kinase (RTK) receptor.

5. The targeted micelle active agent carrier of claim 4, wherein the targeting moiety is a platelet-derived growth factor (PDGF) peptide or an epidermal growth factor (EGF) peptide.

6. The targeted micelle active agent carrier of claim 1, wherein the active agent is a chemotherapeutic agent.

7. The targeted micelle active agent carrier of claim 6, wherein the chemotherapeutic agent is temozolomide.

8. The targeted micelle active agent carrier of claim 1, wherein the micellar structure further comprises a phosphatidylcholine lipid and cholesterol.

9. The targeted micelle active agent carrier of claim 1, wherein the targeting moiety and the micellar structure are present at a molar ratio of about 0.005 to about 0.01 (targeting moiety: micellar structure).

10. A method of treating a brain cancer in a patient, comprising:
administering the targeted micelle active agent carrier of claim 1 to the patient, wherein the targeted micelle active agent carrier crosses the blood-brain barrier to target the brain cancer and deliver the active agent, thereby treating the brain cancer.

11. The method of claim 10, wherein the brain cancer is a glioblastoma.

12. A targeted micelle active agent carrier, comprising:
a. a micellar structure comprising poly(ethylene glycol)-phosphatidylethanolamine-amine (PEG-PE-amine) and D-α-tocopheryl polyethylene glycol succinate,
b. a targeting moiety associated with the PEG-PE-amine; and
c. an active agent encapsulated within the micellar structure.

13. The targeted micelle active agent carrier of claim 12, wherein the targeting moiety targets a receptor tyrosine kinase (RTK) receptor.

14. The targeted micelle active agent carrier of claim 13, wherein the targeting moiety is a platelet-derived growth factor (PDGF) peptide or an epidermal growth factor (EGF) peptide.

15. The targeted micelle active agent carrier of claim 12 wherein the active agent is a chemotherapeutic.

16. The targeted micelle active agent carrier of claim 15, wherein the chemotherapeutic is temozolomide.

17. The targeted micelle active agent carrier of claim 12, wherein the micellar structure further comprises a phosphatidylcholine lipid and cholesterol.

18. The targeted micelle active agent carrier of claim 12, wherein the targeting moiety and the micellar structure are present at a molar ratio of about 0.005 to about 0.01 (targeting moiety:micellar structure).

19. Use of the targeted micelle active agent carrier of claim 12, in the treatment of a glioblastoma in a patient, wherein the targeted micelle active agent carrier crosses the blood-brain barrier to target the glioblastoma and deliver the active agent.

20. The use of claim 19, wherein the targeting moiety is a platelet-derived growth factor (PDGF) peptide and wherein the targeting moiety and the micellar structure are present at a molar ratio of about 0.008 to about 0.01 (targeting moiety:micellar structure).
